(19) United States
(12) Patent Application Publication
(10) Pub. No.: US 2021/0322731 A1
(43) Pub. Date: Oct. 21, 2021

(54) MEDICAL GUIDEWIRE ASSEMBLY HAVING IDENTIFICATION DEVICE

(71) Applicant: Baylis Medical Company Inc., Montreal (CA)

(72) Inventors: John Paul Urbanski, Toronto (CA); Christian Balkovec, Kitchener (CA)

(21) Appl. No.: 17/348,939

(22) Filed: Jun. 16, 2021

Related U.S. Application Data
(63) Continuation-in-part of application No. 16/749,224, filed on Jan. 22, 2020.

(51) Int. Cl. A61M 25/09; A61B 17/34

(52) U.S. Cl. CPC A61M 25/09; A61B 17/3478; A61M 25/0108; A61M 2025/09166; A61M 2025/09183

(57) ABSTRACT

A medical guidewire configured for puncturing a tissue. The medical guidewire includes an elongate guidewire body having a proximal portion with a first outer diameter and a distal portion with a second outer diameter. The second outer diameter is smaller than the first outer diameter, forming a tapered section between the first outer diameter and second outer diameter. The medical guidewire further includes a piercing device extending from the distal portion, forming a puncturing distal tip. An identification device is positioned on the elongate guidewire body on the first outer diameter proximal the tapered section. The identification device is configured to enhance detectable visibility of the medical guidewire.

### CROSS-REFERENCE TO RELATED APPLICATIONS

The present application is a continuation-in-part of U.S. application Ser. No. 16/749,224 filed on Jan. 22, 2020.

### TECHNICAL FIELD

This document relates to the technical field of (and is not limited to) a medical guidewire assembly including a piercing stylet device and an identification device configured to enhance visibility of the piercing stylet device (and method therefor).

### BACKGROUND

Known medical devices, such as a medical guidewire assembly, are configured to facilitate a medical procedure, and help healthcare providers diagnose and/or treat medical conditions of patients.

### SUMMARY

It will be appreciated that there exists a need to mitigate (at least in part) at least one problem associated with the known (existing) medical guidewire assemblies (also called the existing technology). After much study of, and experimentation with, the existing medical guidewire assemblies, an understanding (at least in part) of the problem and its solution have been identified (at least in part) and are articulated (at least in part) as follows:

The following describes problem(s) associated with known medical guidewire assemblies: piercing stylets for transseptal puncture are typically characterized by having an extremely sharp distal tip that enhances tissue puncture efficacy and lowers the required input force compared to sharpened hypotubes. A hypotube is a long metal tube with micro-engineered features along its length. The lower force input to puncture the fossa ovalis in the heart is facilitated by using thin, ductile materials with a pointed bevel at the distal tip. The device is able to be shaped to a configuration where this sharp distal tip is pointed away from the leading edge when the device is in its relaxed configuration. Since elastic materials are used, the device shape can be temporarily manipulated and straightened through accessory devices in order to puncture the fossa ovalis with the sharp distal tip, but then it returns to its relaxed configuration with the distal tip pointing away from the leading edge once outside an accessory device.

Known imaging modalities used by physicians in transseptal puncture and catheterization procedures include fluoroscopy and echocardiography, more specifically, Intracardiac Echocardiography (ICE) and Transesophageal Echocardiography (TEE). Fluoroscopy relies on directing an x-ray beam through the body where the signal is attenuated by tissues and used to create a series of real-time images rather than a static view typical of traditional x-rays. Echocardiography relies on using ultrasound waves to reflect off mediums (such as tissue, catheters, etc.) to create an image of their surroundings. In order to stand out and be easily visualized under either of these imaging modalities, an object needs a density that is higher than the surrounding structures, or in the case of echocardiography, the object (the target for imaging) needs to create sufficient interference to overcome the interference already created by tissue, blood, other catheters/devices, etc.

For instance, a disadvantage of a relatively smaller cross-section material and/or a relatively lower density material to be used for creating a piercing stylet (for transseptal puncturing purposes) is that the leading edge of the piercing stylet is difficult to see via typical imaging modalities. They do not sufficiently attenuate x-rays, or generate enough interference, making them less visible than materials of higher atomic number. Nitinol, for example, is a typical shape-memory alloy used for this type of application as it readily adopts an applied shape which resists permanent alteration and is highly kink resistant. Despite these advantages, nitinol is not a particularly radiopaque material. While more dense materials can be added to the body of the device to enhance visibility, the leading/most distal edge remains challenging to visualize. Being able to identify the precise location of the leading edge is important for navigating to an appropriate location within the desired anatomy. Further, being able to better identify the sharp distal tip at the moment of puncture would be highly advantageous for increasing confidence and puncture site accuracy. If the device cannot be seen, or is too faint, there is a higher potential for error and procedural delay. Further, in the case of piercing stylets, lack of radio-visibility increases the chances of the sharp tip contacting unintended anatomical structures and causing damage.

For instance, some known medical guidewire assemblies greatly decrease the mechanical input force required to puncture the tissue. They are made primarily of nitinol, a lightweight shape-memory alloy of Nickel and Titanium that resists kinking. They have tungsten coils on a portion of the wire body which enhances visibility of that section, but the leading/most distal edge of the distal curve remains difficult to visualize under conventional medical imaging techniques given that nitinol is not particularly radiopaque, and the cross-section is very small. As a result, the leading edge of these devices floats freely in the left atrium after transseptal puncture without adequate feedback of where the sharp distal tip is. Rather, the physician must infer the location based on the radiopaque section on the guidewire body. Further, during the moment of puncture of the fossa ovalis, when the sharp distal tip is at the leading edge, the actual crossing site cannot be visualized, but must be inferred by the “tenting” effect the accessory device has on the tissue.

Furthermore, as previously mentioned, guidewires are designed with a smaller outer diameter at the leading distal end compared to the main guidewire body. This is so that the guidewire enables the distal portion to be floppier and, thus, less traumatic to vasculature it encounters. However, the floppier distal portion is not ideal for facilitating the exchange of therapy devices; rather, the larger outer diameter of the main guidewire body is ideal for providing the support required to steer and deliver end therapy devices to the left atrium.

It would be desirable to provide an identification device mounted to a medical guidewire assembly. The identification device is spaced apart from a piercing stylet device of the medical guidewire assembly. The identification device is configured to enhance the detectible visibility of the piercing stylet device.

It would be desirable to provide the user with the ability to determine when the larger (maximum) outer diameter of the main body is positioned adequately to provide support for facilitating the exchange of therapy devices. The identification device is configured to provide users with visualization of the maximum outer diameter of the main guidewire body. The identification device would provide users with the ability to determine when the guidewire has been advanced into the left atrium and have achieved the optimal positioning to support and facilitate the exchange of therapy devices.

To mitigate, at least in part, at least one problem associated with the existing technology, there is provided a medical guidewire configured for puncturing a tissue of the heart. The medical guidewire has an elongate guidewire body which has a proximal portion of a first outer diameter and a distal portion with a second outer diameter. The first outer diameter is larger than the second outer diameter, forming a tapered section. A piercing device extends from the distal portion, forming a puncturing distal tip. An identification device is positioned on the elongate guidewire body, at the first outer diameter, proximal to the tapered section. The identification device is configured to enhance detectable visibility of the medical guidewire.

In some embodiments of the present invention, the elongate guidewire body comprises a plurality of identification devices. The plurality of identification devices are separated from one another at a distance between about 0.1 mm to about 20 mm, preferably at about 1.25 mm.

In some embodiments of the present invention, the plurality of identification devices forms a discrete grouping. In some embodiments, the plurality of identification devices are separated from one another at a distance between about 0.1 mm to about 20 mm, preferably about 1.25 mm, and the discrete groupings are separated from one another at a distance between about 1 mm to about 40 mm, preferably about 10 mm.

In some embodiments of the present invention, the width of the identification device can be about 2.5 mm.

In some embodiments of the present invention, the identification device is an echogenic element. The echogenic element may be comprised of surface irregularities on a section of the elongate guidewire body. The surface irregularities may be formed by laser etching, centerless grinding, or abrasive blasting, or any combination thereof.

In an alternative embodiment of the present invention, the identification device is a radiopaque element. In some embodiments, the radiopaque element is a radiopaque coil. The radiopaque element may also be formed by deposition of dense materials that attenuate x-rays via thermal spraying, electroplating, pad printing, or vapor deposition, or any combination thereof.

In some embodiments of the present invention, the identification device may be a combination of an echogenic element and a radiopaque element.

To mitigate, at least in part, at least one problem associated with the existing technology, there is provided a method for accessing the left atrium of a heart. The method comprises the steps of advancing a medical guidewire into a right atrium of the heart and positioning a puncturing distal tip of the medical guidewire on a target location of a septum, the medical guidewire has an elongate guidewire body, wherein the elongate guidewire body has a proximal portion with a first outer diameter, a distal portion with a second outer diameter, where the second outer diameter is smaller than the first outer diameter, forming a tapered section. A piercing device extending from the distal portion, forming the puncturing distal tip. An identification device positioned on the elongate guidewire body, with the identification device being located on the first outer diameter proximal the tapered section. A step of puncturing the septum and advancing the distal portion of the medical guidewire into the left atrium while visualizing the identification device on a medical imaging system. A step of advancing the medical guidewire into the left atrium until the identification device has reached the septum, whereby the medical guidewire is in a position to facilitate and support an exchange of an ancillary device. In some embodiments, the identification device is either a radiopaque element or an echogenic element or a combination of the radiopaque element and the echogenic element. In some embodiments, the medical imaging system is a fluoroscopy medical-imaging system and/or an echocardiography medical-imaging system.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 depicts a side view of an embodiment of a medical guidewire assembly; and

FIG. 2 depicts a side view of an embodiment of the medical guidewire assembly of FIG. 1; and

FIG. 3 depicts a side view of an embodiment of the medical guidewire assembly of FIG. 1; and

FIG. 4 depicts a side view of an embodiment of the medical guidewire assembly of FIG. 1; and

FIG. 5 depicts a table of configuration options for the medical guidewire assembly of FIG. 1; and

FIG. 6 depicts a side view of an embodiment of a medical guidewire assembly; and

FIG. 7 depicts a side view of an alternative embodiment of a medical guidewire assembly; and

FIG. 8 depicts a side view of an alternative embodiment of a medical guidewire assembly; and

FIG. 9 depicts a side view of the medical device assembly of FIG. 7 advanced through a puncture formed in the tissue of a heart; and

FIG. 10 to FIG. 12 depicts the method of use with an embodiment of the medical guidewire assembly depicted in FIG. 6 to FIG. 8.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

medical guidewire assembly 100
flexible distal shaft section 102
spatial geometry 103
distal tip portion 104
first leading distal portion 106
second leading distal portion 108
stylet device 110
identification device 112
radiopaque element 114
echogenic element 116
larger outer diameter 118
taper section 120
smaller outer diameter 122
identification device grouping 124
puncture 126
left atrium 128
right atrium 130
first identification device 201
second identification device 202
first curved portion 302
second curved portion 304
extension portion 306
table 500
medical imaging system 900
exit portal 901
guidewire introducer 902
direction 903
patient 905
interior longitudinal channel 906
radiopaque sensor 914
echogenic sensor 916

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the claim is defined by the claims (in which the claims may be amended during patent examination after the filing of this application). For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the invention is limited to the subject matter provided by the claims, and that the invention is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

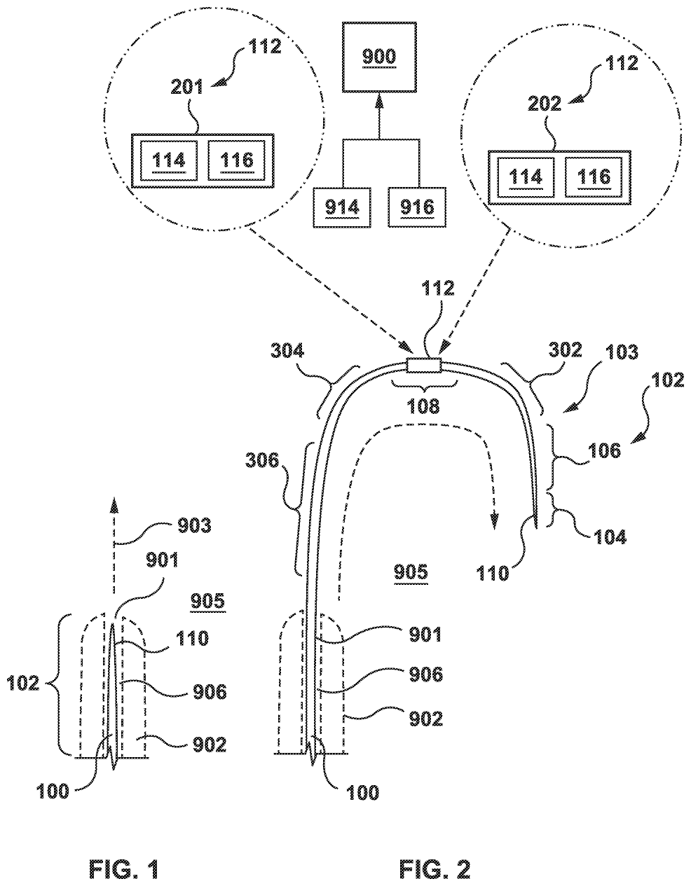

FIG. 1 depicts a side view of an embodiment of a medical guidewire assembly 100 (in which the medical guidewire assembly 100 is depicted in a non-deployed condition as depicted in FIG. 1).

FIG. 2 depicts a side view of an embodiment of the medical guidewire assembly 100 of FIG. 1 (in which the medical guidewire assembly 100 is depicted in a deployed condition as depicted in FIG. 2).

In accordance with the embodiment as depicted in FIG. 1 and FIG. 2, an apparatus includes and is not limited to (comprises) a medical guidewire assembly 100. The medical guidewire assembly 100 is movable along (from) an interior longitudinal channel 906 of the guidewire introducer 902 via an exit portal 901 of the guidewire introducer 902 (such as, along a direction 903 aligned along an axis extending from the exit portal 901 of the guidewire introducer 902). The guidewire introducer 902 and the medical guidewire assembly 100 are each configured to be inserted into a confined space defined by a patient 905. The apparatus further includes a piercing stylet device 110 extending from a distal portion (section) of the medical guidewire assembly 100. The piercing stylet device 110 is configured to physically cut the tissue of the patient 905 in response to removal of the medical guidewire assembly 100 from the guidewire introducer 902 via the exit portal 901 toward the tissue of the patient 905 (this is done, preferably, after the guidewire introducer 902 and the medical guidewire assembly 100 have been inserted into the confined space defined by the patient 905).

In accordance with the embodiment as depicted in FIG. 1 and FIG. 2, the piercing stylet device 110 (also called a sharp distal tip) is configured to (preferably) mechanically puncture the tissue (such as, the fossa ovalis in the heart) of the patient 905. It will be appreciated that sharpness may be a subjective term. It will be appreciated that every tissue eventually fails (that is, may be punctured) when a load (applied force) exceeds the tolerance level of the tissue. Preferably, the piercing stylet device 110 is configured to mechanically puncture the tissue (such as the fossa ovalis) under an applied force (a force applied to the piercing stylet device 110). The piercing stylet device 110 may include a blunt radiofrequency tip (known and not depicted). More dense materials are able to be used for construction of the piercing stylet device 110. It will be appreciated that the piercing stylet device 110 does not necessarily have to be literally a “sharp” tool but it is configured, at a minimum, to puncture a hole through tissue.

In accordance with the embodiment as depicted in FIG. 2, the apparatus further includes an identification device 112 mounted to the medical guidewire assembly 100. The identification device 112 is spaced apart from the piercing stylet device 110. The identification device 112 is configured to enhance the detectible visibility of the piercing stylet device 110 by a medical imaging system 900. The identification device 112 is configured to detect a spatial position of the piercing stylet device 110 (preferably, after the piercing stylet device 110 is deployed or removed from the interior of the medical guidewire assembly 100). The identification device 112 is configured to emit (transmit, provide) a sensed position signal indicating the presence of (the detection of a position of) the piercing stylet device 110 (preferably, after the piercing stylet device 110 is deployed (removed) from the medical guidewire assembly 100). The identification device 112 is configured to be sensed by the medical imaging system 900 (so that then the medical imaging system 900 may compute the spatial position of the identification device 112 and display the spatial position of the identification device 112 on a display device (known and not depicted) to a doctor. Once the medical imaging system 900, in use, determines (computes) the sensed position of the identification device 112, the medical imaging system 900 is configured to process and compute the position of the identification device 112, and to then display the computed position of the identification device 112 (and/or the position of the piercing stylet device 110) on a display screen (known and not depicted); this is done preferably after the piercing stylet device 110 is deployed or removed from the medical guidewire assembly 100. Examples of the medical imaging system 900 may include fluoroscopy medical-imaging systems, echocardiography medical-imaging systems, etc., and any equivalent thereof. Examples of the identification device 112 are described below. In accordance with an alternative embodiment, the medical imaging system 900 is configured to transmit (convey) a signal to the identification device 112. The identification device 112 is configured to be differentiated from surrounding tissue (structures) by attenuating and/or scattering the signal sent out by the medical imaging system 900 toward the identification device 112 (in a manner that exceeds the attenuation or scattering of the surrounding structures). The identification device 112 may appear more visually distinct to an observer when the observer views the medical image rendered by the medical imaging system 900.

In accordance with the embodiment as depicted in FIG. 2, the medical guidewire assembly 100 provides a piercing stylet device 110 with an identification device 112 positioned on the medical guidewire assembly 100. There are options described (below) for the spatial positioning of the identification device 112 on the medical guidewire assembly 100 relative to the piercing stylet device 110.

The identification device 112 enhances the visibility of the piercing stylet device 110 to the doctor (physician) by way of a medical imaging system 900 (such as fluoroscopy, echocardiography, etc.). The medical guidewire assembly 100 allows the doctor to visualize where critical sections of the piercing stylet device 110 may be located (spatially positioned) inside the patient 905, thereby ensuring greater (improved) overall control or manipulation of the piercing stylet device 110, enhanced puncturing accuracy, and/or reduced risk of unintended tissue damage to the patient 905. In some embodiments, the identification device 112 includes echogenic elements; for other embodiments, the identification device 112 includes radiopaque elements, etc.

In accordance with the embodiment as depicted in FIG. 2, there are several possible uses for the medical guidewire assembly 100. The medical guidewire assembly 100 enhances, at least in part, visibility of a physical aspect of the medical guidewire assembly 100, such as a leading edge of a distal curve of the medical guidewire assembly 100 and/or the piercing stylet device 110. For instance, the piercing stylet device 110 may be configured for transseptal catheterization procedures under medical imaging modalities such as fluoroscopy and echocardiography, etc. This may provide the benefit of visual feedback to a physician during procedures as to the location of the piercing stylet device 110 while the medical guidewire assembly 100 is positioned inside the patient 905, thereby reducing procedural uncertainty, reducing overall procedure time, and/or increasing the safety of the use of the piercing stylet device 110.

In accordance with the embodiment as depicted in FIG. 1 and FIG. 2, there is provided a method of enhancing detectible visibility of a piercing stylet device 110 of a medical guidewire assembly 100 that is movable along an interior longitudinal channel 906 of the guidewire introducer 902 via an exit portal 901 of the guidewire introducer 902/The guidewire introducer 902 and the medical guidewire assembly 100 are each configured to be inserted into a confined space defined by a patient 905. The piercing stylet device 110 extends from a distal portion of the medical guidewire assembly 100. The piercing stylet device 110 is configured to physically cut the tissue of the patient 905 in response to removal of the medical guidewire assembly 100 from the guidewire introducer 902 and toward the tissue of the patient 905 (after the guidewire introducer 902 and the medical guidewire assembly 100 have been inserted into the confined space defined by the patient 905). An identification device 112 is mounted to the medical guidewire assembly 100. The identification device 112 is spaced apart from the piercing stylet device 110. The method includes and is not limited to (comprises) a synergystic combination of (A) moving the medical guidewire assembly 100 through the guidewire introducer 902, and (B) using the identification device 112 to enhance the detectible visibility of the piercing stylet device 110 by a medical imaging system 900. Detectible visibility includes any detection by any suitable sensor having a sensitivity tolerance.

In accordance with the embodiment as depicted in FIG. 2, the identification device 112 includes (for instance) a radiopaque element 114 configured to be detectable by a radiopaque sensor 914 of a medical imaging system 900. The radiopaque element 114 includes a substance that is opaque to X-rays or radiation (that is, impenetrable to X-rays and other radiation). The radiopaque element 114 may include a radiopaque coating and/or a radiopaque element, and may be positioned, for instance, on the medical guidewire assembly 100 (such as a distal curve) and/or near the piercing stylet device 110 (also called the sharp distal tip). A radiopaque coating deposits element with a high density such as gold, tungsten, or platinum onto the nitinol material of the medical guidewire assembly 100. This may be facilitated by any suitable technique, such as that described in United States Patent Publication Number US20070106374A1 and/or PCT Patent Application Number WO2005122961A3. Such coatings are thin enough on the surface of the medical guidewire assembly 100 so as not to significantly alter the profile of the medical guidewire assembly 100 and introduce a reduction in fossa ovalis crossing efficacy. Crossing the septum with the medical guidewire assembly 100 may remain smooth and increase the visibility of the specified sections under imaging modalities. The following is a list of techniques used to apply radiopaque coatings to medical device surfaces: (A) dip coating can be performed with certain materials where the radiopaque elements are solvent-based, the relevant sections are simply dipped into the coating and allowed to bond to the surface; (B) pad printing is another technique for application of these coatings where a silicone pad is used to transfer the aqueous material onto a desired substrate; (C) screen printing is where a mesh is used to transfer a solvent/ink onto a substrate material; (D) the solvent is allowed to bond with the substrate; (E) syringe dispensation is used to directly deposit a solvent-based radiopaque material to the desired surface using a syringe to direct the flow and placement; (F) Physical Vapor Deposition (PVD) refers to any technique in which a material is vaporized and condenses onto a target surface to form a coating thereon; (G) electroplating including usage of an electric current to deposit dissolved metal cations onto a surface; and/or (H) any processes for depositing a radiopaque materials onto any type of compatible surface. A coating may include a radiopaque element 114 deposited on a surface of a metal braid and/or a surface of a polymer, etc., and any equivalent thereof. The radiopaque element 114 does not need to be a coating, but rather, may be any material with a relatively higher atomic number to cause attenuation of the fluoroscopic and/or x-ray spectrum at the specified critical sites. Other embodiments of the radiopaque element 114 may include the echogenic elements or the radiopaque elements exclusively, etc. The radiopaque element 114 may include a radiopaque coating; for instance, any method that adds elements of a sufficiently higher atomic number may be utilized to attenuate x-rays to a greater extent than the underlying substrate on the leading distal edge located near the piercing stylet device 110, etc. An option for the radiopaque element 114 may include making the distal curve of a radiopaque material such as platinum, gold, or tungsten (instead of the underlying substrate being a non-radiopaque material with a more radiopaque material bonded to the surface). Another option for the radiopaque element 114 may include using a radiopaque coil placed over the distal curve (of the medical guidewire assembly 100) which may enhance radiopacity and/or echogenicity. With a mechanical puncture device, a smooth transition may need to be created over the section of the curve with the coil to enable smooth crossing of the medical guidewire assembly 100 across the septum. This could be achieved via a PTFE liner placed over top of the coil or via a ramp of material built up in front of and behind the coil. Yet another option for the radiopaque element 114 may include creating the medical guidewire assembly 100 out of a spring-tempered stainless steel mandrel that may also enhance radiopacity and/or echogenicity of the medical guidewire assembly 100 (while not as radiopaque as tungsten, stainless steel may improve upon the visibility of nitinol).

In accordance with the embodiment as depicted in FIG. 2, the identification device 112 includes (for instance) an echogenic element 116 configured to be detectable by an echogenic sensor 916 of a medical imaging system 900. The echogenic element 116 includes a substance having the ability to bounce an echo (such as, to return a signal in ultrasound examinations). The echogenic element 116 may include (for instance) laser etching (laser etched lines or grooves) positioned on a selected portion (position) of the medical guidewire assembly 100 (such as, on distal curve and/or near the piercing stylet device 110). Relatively smaller etches may be formed or created on the distal curve around the apex and within 1.0 millimeters (mm) of the start of a bevel section of the piercing stylet device 110. The laser etched lines (or etched grooves) may introduce at least some surface irregularity to the surface of the medical guidewire assembly 100, and/or may generate interference under echocardiography, thereby enhancing the visibility of these sections of the medical guidewire assembly 100. The echogenic element 116 may include surface irregularities. The surface irregularities do not need to be created via laser-etching. It will be appreciated that any method which creates a surface irregularity that enhances the contrast of a portion of the medical guidewire assembly 100 (such as the distal curvature) under echocardiography may be suitable. The echogenic element 116 may include laser-etching formed on a surface of the medical guidewire assembly 100. Any method that creates a surface irregularity on a portion of the medical guidewire assembly 100 (such as the distal leading edge of the medical guidewire assembly 100) may be used. The echogenic element 116 may include laser-etching lines (the irregularity does not need to be a line, but rather, can be any shape to generate interference under echocardiography imaging). Other methods to create the echogenic marker may include abrasive blasting (propelling of abrasive material under high pressure) or centerless grinding (utilization of a grinding and regulating wheel to remove material from the surface). The echogenic element 116 may include an ultrasound transducer incorporated into a position on the medical guidewire assembly 100 such as the tip, such that the echogenic element 116 is able to emit ultrasound signals and create interference for visualization under echocardiography imaging. The echogenic element 116 may provide (include) surface irregularities and/or the addition of elements on a portion of the medical guidewire assembly 100 (such as the distal curve of the medical guidewire assembly 100) of sufficiently high atomic number so as to attenuate x-rays to a greater degree than the underlying device material.

In accordance with the embodiment as depicted in FIG. 2, the identification device 112 includes (for instance) a synergistic combination of: (A) a radiopaque element 114 configured to be detectable by a radiopaque sensor 914 of a medical imaging system 900, and (B) an echogenic element 116 configured to be detectable by an echogenic sensor 916 of a medical imaging system 900.

In accordance with the embodiment as depicted in FIG. 2, the medical guidewire assembly 100 includes (preferably) a synergistic combination of (for instance): (A) a distal tip portion 104 (the piercing stylet device 110 that extends from the distal tip portion 104), (B) a first leading distal portion 106 that is positioned proximate to the distal tip portion 104, and (C) a second leading distal portion 108 that is spaced apart from the first leading distal portion 106.

In accordance with the embodiment as depicted in FIG. 2, the medical guidewire assembly 100 includes (preferably) a flexible distal shaft section 102 that is movable, at least in part, through the guidewire introducer 902.

The flexible distal shaft section 102 includes a distal tip portion 104. The piercing stylet device 110 extends from the distal tip portion 104 of the flexible distal shaft section 102. For instance, the piercing stylet device 110 is configured to cut or puncture tissue (a biological wall) of the patient 905 in response to movement of the flexible distal shaft section 102 through the guidewire introducer 902 and toward the tissue of the patient 905. The flexible distal shaft section 102 is configured to have a predetermined spatial geometry 103 once the flexible distal shaft section 102 is removed, at least in part, from the guidewire introducer 902. The predetermined spatial geometry 103 is a geometry (shape) of the flexible distal shaft section 102 that is formed (or repeatably formed every time) once the flexible distal shaft section 102 is removed (at least in part) from the interior of the guidewire introducer 902. The predetermined spatial geometry 103 is a geometry (shape) of the flexible distal shaft section 102 that is a relaxed formation that is unsupported by the interior of the guidewire introducer 902.

In accordance with the embodiment as depicted in FIG. 2, the medical guidewire assembly 100 (or the flexible distal shaft section 102) may include a nitinol guidewire body. The primary material that the medical guidewire assembly 100 may be composed of is nitinol. It is a shape memory that may be manipulated and deformed followed by a return to the original shape it was set in. The medical guidewire assembly 100 may be able to be placed inside of stiff accessory devices with a hollow lumen. The guidewire introducer 902 (also called an accessory device) may be configured to straighten the medical guidewire assembly 100 so that the piercing stylet device 110 may be used to puncture the fossa ovalis (tissue). Following the puncture of the tissue, the medical guidewire assembly 100 is advanced and is able to return to its relaxed shape where the piercing stylet device 110 points away from the leading edge of the medical guidewire assembly 100. The medical guidewire assembly 100 may have an outer diameter that is less than (preferably) about 0.032 inches. The medical guidewire assembly 100 includes (preferably a nitinol guidewire body. However, the medical guidewire assembly 100 may include any material where the medical guidewire assembly 100 may be manipulated and returned to a relaxed, curved configuration (as depicted in FIG. 2).

In accordance with the embodiment as depicted in FIG. 2, the medical guidewire assembly 100 is configured to be inserted into a confined space defined by a patient 905. The medical guidewire assembly 100 includes (preferably) a relatively thin and flexible wire (an elongated flexible shaft) configured to be inserted into a confined or tortuous space (such as the confined space defined by the patient 905). The medical guidewire assembly 100 includes, preferably, a flexible tube (made from a medical grade material) configured to be inserted through a narrow opening into a body cavity space (the confined space defined by the patient 905). The medical guidewire assembly 100 is (preferably) impermeable by a bodily fluid located in the confined space defined by the living body 902 (once the flexible medical guidewire assembly 102 is inserted into the confined space defined by the patient 905). The medical guidewire assembly 100 includes, preferably, SAE (Society of Automotive Engineering) Type 304 Stainless Steel. SAE Type 304 stainless steel contains both chromium (from between 15% to 20%) and nickel (between 2% to 10.5%) metals as the main non-iron constituents. The medical guidewire assembly 100 includes (in accordance with another option) superelastic nitinol. Nitinol alloys exhibit two closely related and unique properties: shape memory effect (SME) and superelasticity (SE; also called pseudoelasticity or PE). Shape memory is the ability of nitinol to undergo deformation at one temperature, then recover its original, undeformed shape upon heating above its transformation temperature. Superelasticity occurs at a narrow temperature range just above its transformation temperature; in this case, no heating is necessary to cause the undeformed shape to recover, and the material exhibits enormous elasticity, from about ten (10) to thirty (30) times that of ordinary metal. The medical guidewire assembly 100 includes (in accordance with a preferred embodiment) bio-compatible materials properties suitable for sufficient performance properties (dielectric strength, thermal performance, insulation and corrosion, water and heat resistance) for safe performance to comply with industrial and regulatory safety standards (or compatible for medical usage). Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

In accordance with the embodiment as depicted in FIG. 2, the identification device 112 is mounted to the flexible distal shaft section 102. The identification device 112 is spaced apart from the piercing stylet device 110. The identification device 112 is configured to enhance the detectible visibility of the piercing stylet device 110.

In accordance with the embodiment as depicted in FIG. 2, the flexible distal shaft section 102 includes (preferably) a synergistic combination of (for instance): (A) a distal tip portion 104 (the piercing stylet device 110 that extends from the distal tip portion 104 of the flexible distal shaft section 102), (B) a first leading distal portion 106 that is positioned proximate to the distal tip portion 104, and (C) a second leading distal portion 108 that is spaced apart from the leading distal portion 106.

In accordance with the embodiment as depicted in FIG. 2, the flexible distal shaft section 102 includes a synergistic combination of (for instance): (A) a distal tip portion 104 (the piercing stylet device 110 extends from the distal tip portion 104), (B) a first leading distal portion 106 positioned proximate to the distal tip portion 104, (C) a first curved portion 302 extending from the distal tip portion 104, (D) a second leading distal portion 108 extending from the first curved portion 302, and the second leading distal portion 108 is spaced apart from the leading distal portion 106, (E) a second curved portion 304 extending from the second leading distal portion 108, and (F) an extension portion 306 configured to extend between the second curved portion 304 and the guidewire introducer 902. It will be appreciated that the length of the flexible distal shaft section 102 may be a continuous curved section or a discontinuous section or a combination thereof.

In accordance with the embodiment as depicted in FIG. 2, the identification device 112 is mounted to the second leading distal portion 108 of the flexible distal shaft section 102.

In accordance with the embodiment as depicted in FIG. 2, an apparatus includes and is not limited to (comprises) a medical guidewire assembly 100 that is movable along an interior longitudinal channel 906 of the guidewire introducer 902 (via an exit portal 901 of the guidewire introducer 902). The guidewire introducer 902 and the medical guidewire assembly 100 are each configured to be inserted into a confined space defined by a patient 905. A piercing stylet device 110 extends from a distal portion of the medical guidewire assembly 100. The piercing stylet device 110 is configured to physically cut the tissue of the patient 905 in response to removal of the medical guidewire assembly 100 from the guidewire introducer 902 and toward the tissue of the patient 905 (after the guidewire introducer 902 and the medical guidewire assembly 100 have been inserted into the confined space defined by the patient 905). An identification device 112 is mounted to the medical guidewire assembly 100. The identification device 112 is spaced apart from the piercing stylet device 110. The identification device 112 is configured to enhance the detectible visibility of the piercing stylet device 110 by a medical imaging system 900. The medical guidewire assembly 100 includes (preferably) a flexible distal shaft section 102 being movable, at least in part, through the guidewire introducer 902. The flexible distal shaft section 102 includes a distal tip portion 104. The piercing stylet device 110 extends from the distal tip portion 104 of the flexible distal shaft section 102. The piercing stylet device 110 is configured to puncture the tissue of the patient 905 in response to movement of the flexible distal shaft section 102 through the guidewire introducer 902 and toward the tissue. The flexible distal shaft section 102 is configured to have a predetermined spatial geometry 103 once the flexible distal shaft section 102 is removed, at least in part, from the guidewire introducer 902. The flexible distal shaft section 102 includes (preferably) a distal tip portion 104 (the piercing stylet device 110 extends from the distal tip portion 104). The flexible distal shaft section 102 also includes (preferably) a first leading distal portion 106 that is positioned proximate to the distal tip portion 104. The flexible distal shaft section 102 includes (preferably) a first curved portion 302 extending from the distal tip portion 104. The flexible distal shaft section 102 includes (preferably) a second leading distal portion 108 extending from the first curved portion 302, and the second leading distal portion 108 is spaced apart from the first leading distal portion 106. The flexible distal shaft section 102 includes (preferably) a second curved portion 304 extending from the second leading distal portion 108. The flexible distal shaft section 102 includes (preferably) an extension portion 306 configured to extend between the second curved portion 304 and the guidewire introducer 902. In accordance with a preferred embodiment (as depicted in FIG. 2), the flexible distal shaft section 102 forms a U-shaped formation, or a J-shaped formation, etc., and any equivalent shape.

In accordance with the embodiment as depicted in FIG. 2, the predetermined spatial geometry 103 (also called the curved distal end) has a relaxed form that may adopt a curvature where the piercing stylet device 110 is pointed away from, or surrounded by, other sections of the medical guidewire assembly 100. This arrangement may help to mitigate unintended contact of anatomical structures with the piercing stylet device 110. The predetermined spatial geometry 103 ensures that the piercing stylet device 110 is not the leading edge when the medical guidewire assembly 100 is in its relaxed configuration (that is, when the predetermined spatial geometry 103 is formed as depicted in FIG. 2). The predetermined spatial geometry 103 (curved shape) may be any formation that fits this criterion.

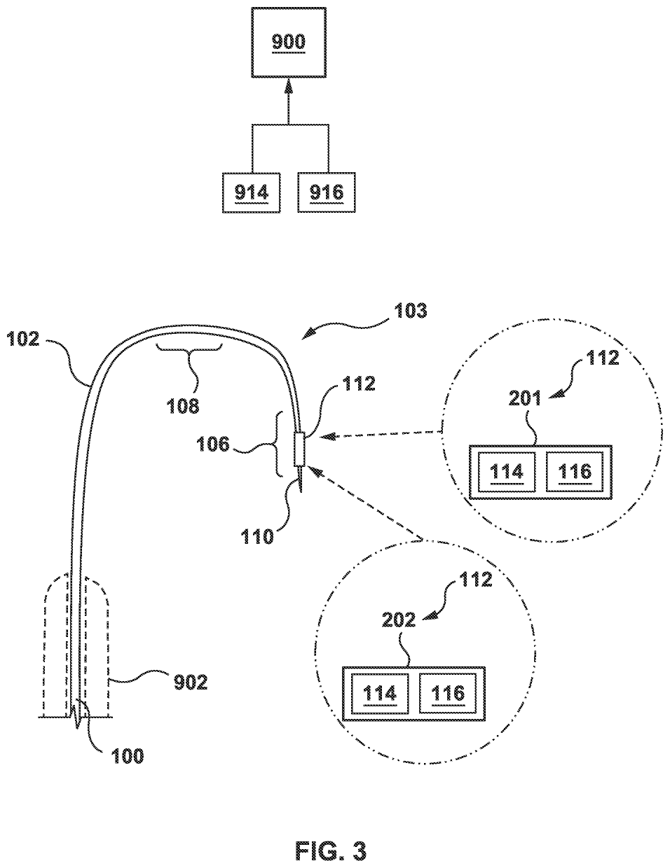

FIG. 3 depicts a side view of an embodiment of the medical guidewire assembly 100 of FIG. 1.

In accordance with the embodiment as depicted in FIG. 3, the identification device 112 is mounted to the distal tip portion 104 (near the piercing stylet device 110).

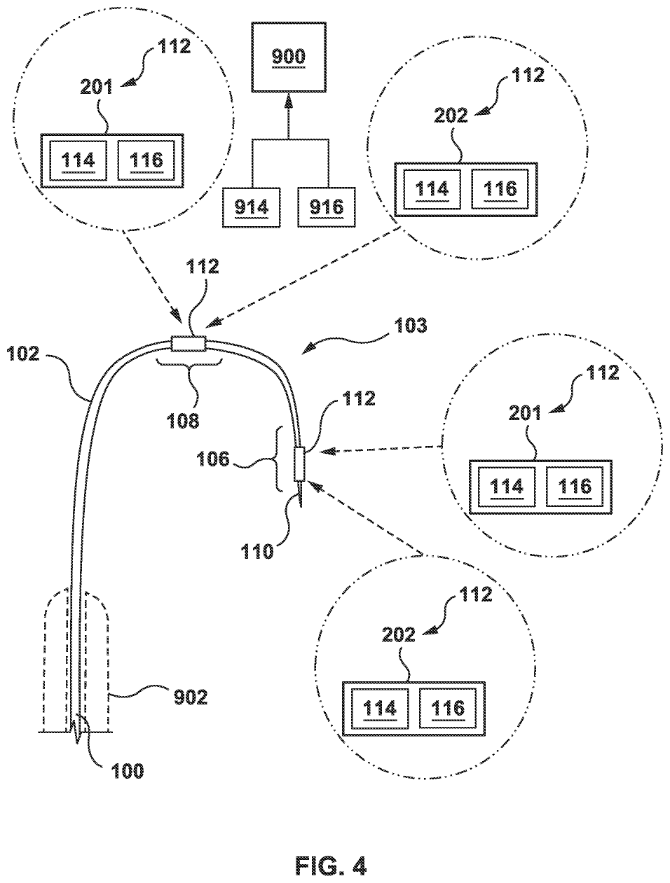

FIG. 4 depicts a side view of an embodiment of the medical guidewire assembly 100 of FIG. 1.

In accordance with the embodiment as depicted in FIG. 4, the identification device 112 includes (preferably) a synergistic combination of: (A) a first identification device 201 mounted to the second leading distal portion 108 of the flexible distal shaft section 102; and (B) a second identification device 202 mounted to the distal tip portion 104 (preferably to enhance visibility of these sections under medical imaging such as fluoroscopy and echocardiography medical-imaging systems).

In accordance with the embodiment as depicted in FIG. 4, the first identification device 201 includes a radiopaque element 114 configured to be detectable by a radiopaque sensor 914 of a medical imaging system 900. The radiopaque sensor 914 is configured to detect signals emitted by the radiopaque element 114. It will be appreciated that the radiopaque element 114 is configured to attenuate the signals emitted by the medical imaging system 900 that is configured to emit radiation. In doing so, the radiopaque element 114 may be visually distinguished from other structures that attenuate the signals to a lesser degree on images rendered by the medical imaging system 900.

In accordance with the embodiment as depicted in FIG. 4, the second identification device 202 includes an echogenic element 116 configured to be detectable by an echogenic sensor 916 of a medical imaging system 900. The echogenic sensor 916 is configured to detect signals emitted by the echogenic element 116. It will be appreciated that the echogenic element 116 is configured to attenuate the signals emitted by the medical imaging system 900 that is configured to emit energy (such as ultrasound waves). In doing so, the echogenic element 116 may be visually distinguished (in use) from other structures that attenuate the signals to a lesser degree on images rendered by the medical imaging system 900.

In accordance with the embodiment as depicted in FIG. 4, the first identification device 201 includes a synergistic combination of: (A) a radiopaque element 114 configured to be detectable by a radiopaque sensor 914 of a medical imaging system 900, and (B) an echogenic element 116 configured to be detectable by an echogenic sensor 916 of a medical imaging system 900. It will be appreciated that the radiopaque element 114 is configured to attenuate the signals emitted by the medical imaging system 900, and the medical imaging system 900 is configured to emit radiation (in doing so, the radiopaque element 114 is (in use) visually distinguished from other structures that attenuate the signals to a lesser degree on the medical images to be rendered by the medical imaging system 900). The echogenic element 116 is configured to attenuate the signals emitted by the medical imaging system 900, and the medical imaging system 900 is configured to emit ultrasound waves (in doing so, the echogenic element 116 is, in use, visually distinguished from other structures that attenuate the signals to a lesser degree on images rendered by the medical imaging system 900).

In accordance with the embodiment as depicted in FIG. 4, the second identification device 202 includes a synergistic combination of: (A) a radiopaque element 114 configured to be detectable by a radiopaque sensor 914 of a medical imaging system 900, and (B) an echogenic element 116 configured to be detectable by an echogenic sensor 916 of a medical imaging system 900. It will be appreciated that the radiopaque element 114 is configured to attenuate the signals emitted by the medical imaging system 900, and the medical imaging system 900 is configured to emit radiation (in doing so, the radiopaque element 114 (in use) is visually distinguished from other structures that attenuate the signals to a lesser degree on the medical images to be rendered by the medical imaging system 900). The echogenic element 116 is configured to attenuate the signals emitted by the medical imaging system 900, and the medical imaging system 900 is configured to emit ultrasound waves (in doing so, the echogenic element 116 is (in use) visually distinguished from other structures that attenuate the signals to a lesser degree on images rendered by the medical imaging system 900).

In accordance with the embodiment as depicted in FIG. 4, the identification device 112 is mounted to any one of, or both of, the second leading distal portion 108 of the flexible distal shaft section 102 and the distal tip portion 104.

In accordance with the embodiment as depicted in FIG. 4, the identification device 112 includes any one of, or both of: (A) a first identification device 201 mounted to any one of, or both of, the second leading distal portion 108 of the flexible distal shaft section 102 and the distal tip portion 104, and (B) a second identification device 202 mounted to any one of, or both of, the second leading distal portion 108 of the flexible distal shaft section 102 and the distal tip portion 104.

In accordance with the embodiment as depicted in FIG. 4, the following is another configuration in which (A) the first identification device 201 includes any one of, or both of, (i) a radiopaque element 114 configured to be detectable by a radiopaque sensor 914 of a medical imaging system 900, and (ii) an echogenic element 116 configured to be detectable by an echogenic sensor 916 of a medical imaging system 900, and (B) the second identification device 202 includes any one of, or both of, (i) a radiopaque element 114 configured to be detectable by a radiopaque sensor 914 of a medical imaging system 900, and (ii) an echogenic element 116 configured to be detectable by an echogenic sensor 916 of a medical imaging system 900. It will be appreciated that the equivalent to (or an option to) an active detection of the radiopaque element 114 and/or the echogenic element 116 by the imaging system 900 may include the radiopaque element 114 and/or the echogenic element 116 being relatively more visually distinguishable from their surroundings.

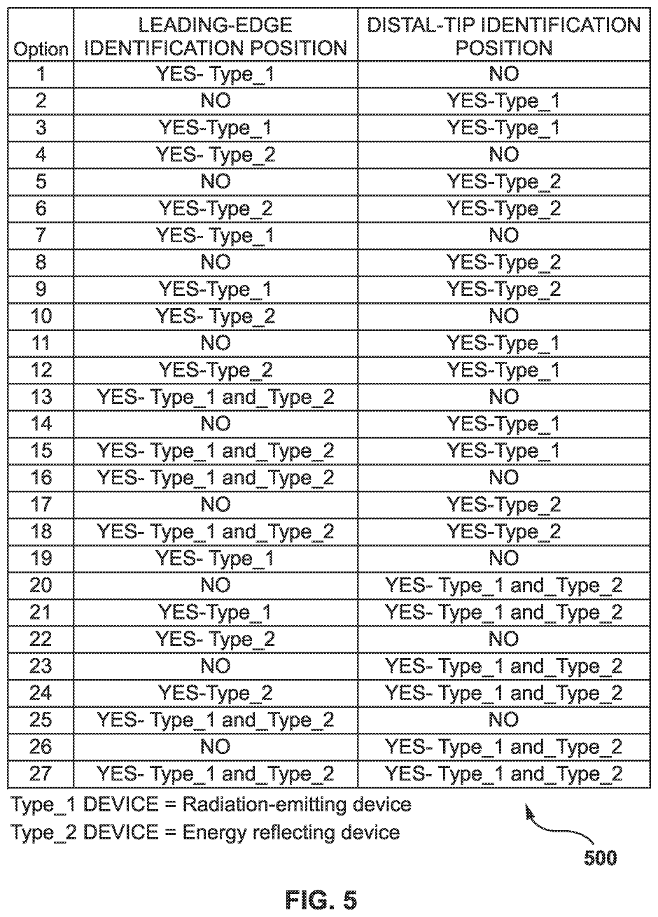

FIG. 5 depicts a table 500 of configuration options for the medical guidewire assembly 100 of FIG. 1.

In accordance with the embodiment as depicted in FIG. 5, the table 500 includes three vertically-aligned columns. The first column shows the option number for each position. The second column shows the possible options for the types of the identification device 112 that may be deployed or positioned at a distal tip identification position (also called the second leading distal portion 108, as depicted in FIG. 2, FIG. 3 and FIG. 4). The third column shows the possible options for the types of the identification device 112 that may be deployed or positioned at the leading-edge identification position (also called the first leading distal portion 106, as depicted in FIG. 2, FIG. 3 and FIG. 4).

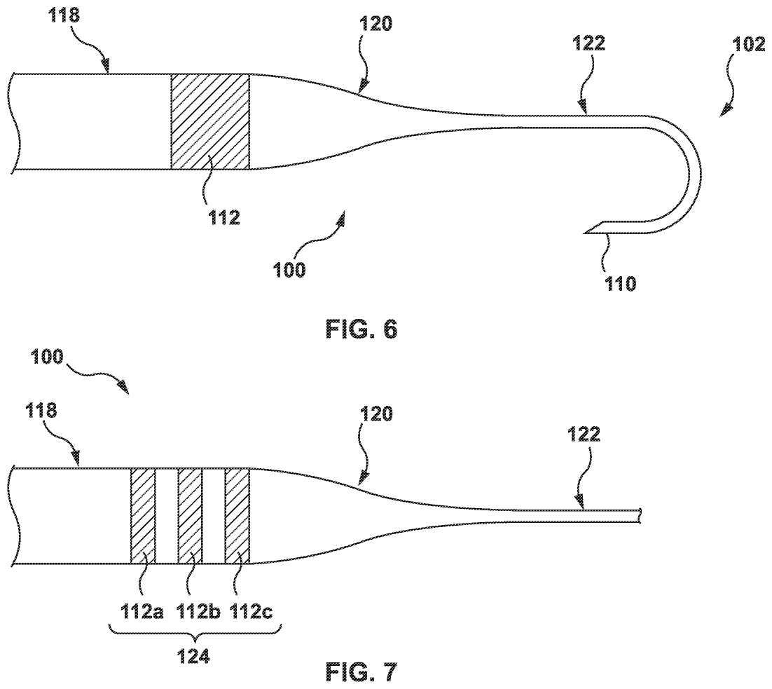

In accordance with the embodiment as depicted in FIG. 6, the identification device 112 may be positioned at the maximum outer diameter 118 of the guidewire assembly 100. As previously described, the medical guidewire assembly 100 comprises a flexible distal shaft section 102. In some embodiments of the present invention, the flexible distal shaft section 102 may be formed by decreasing the outer diameter of the flexible distal shaft section 102; thus, enabling the flexible distal shaft section 102 to be less traumatic to vasculature it makes contact with. This may result in a taper 120 as the diameter moves from a larger outer diameter 118 to a smaller outer diameter 122 of the flexible distal shaft section 102. The larger outer diameter 118 of the guidewire assembly 100 is ideal for providing the support required to steer and deliver therapy devices to the left atrium of the heart. Positioning the identification device 112 at the larger outer diameter 118 of the guidewire assembly 100 would provide users with information that the guidewire assembly has achieved optimal positioning for supporting the exchange of devices. For example, once the flexible distal shaft section 102 has entered the left atrium and the identification device 112 is positioned proximate the puncture site. In another example, the user may anchor the guidewire assembly 100 into the pulmonary vein. In this instance, the user may advance a portion of the smaller outer diameter 122 of the flexible distal shaft section 102 within the vessel. The identification device in this instance may be visible at the puncture site or within the left atrium.

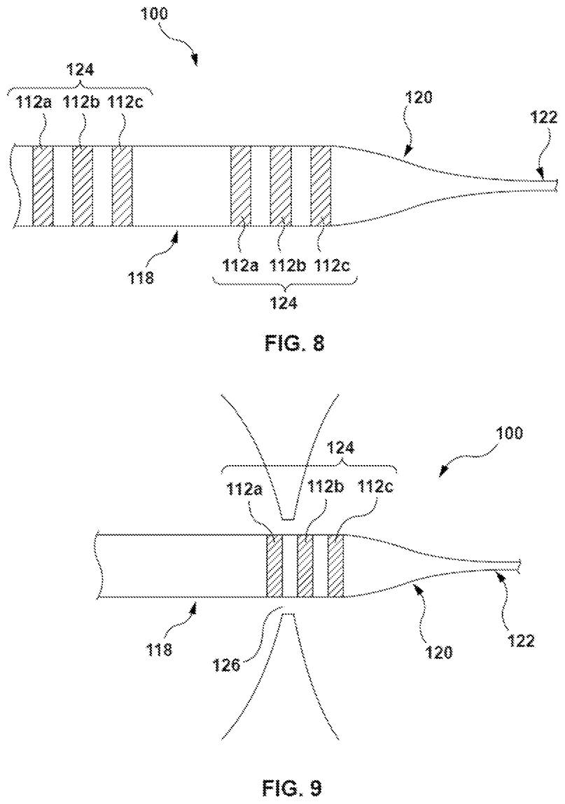

In accordance with the embodiments depicted in FIG. 6, FIG. 7, and FIG. 8, the identification device 112 may be positioned along the guidewire assembly 100, proximal the tapered portion 120. For example, with reference now to FIG. 6, the identification device 112 may be positioned at the beginning of the tapered portion 120 (at the end of the larger outer diameter 118). This position would inform users when the flexible distal shaft section 102 is fully inside the left atrium of a heart and, as such, the guidewire assembly 100 is positioned to provide support for the exchange of devices. In some embodiments, the guidewire assembly 100 may comprise multiple identification devices (112a, 112b, 112c), spaced apart from one another, forming an identification device grouping 124 (as shown in FIG. 7). The identification devices 112a, 112b, and 112c, may be spaced 0.1-20 mm from one another, preferably the spacing is about 1.25 mm; additionally, the identification devices 112a, 112b, and 112c, may have a width between 0.1-20 mm, where the width is preferably about 2.5 mm. In one embodiment, the most distal of the identification devices 112c may be positioned at the beginning of the tapered portion 120. In some embodiments, there may be more than one grouping 124 along the length of the guidewire assembly 100, as depicted in FIG. 8, which may act as depth markers. In some instances, the distance between the groupings 124 may range from 1-40 mm, preferably this distance is about 10 mm. It would be appreciated that a plurality of identification devices 112 may be utilized and a plurality of groupings 124 may be positioned along the length of the guidewire assembly 100.

The identification device 112 may comprise a radiopaque element 114 configured to be detectable by a radiopaque sensor of a medical imaging system. The radiopaque element 114 includes a substance that is opaque to X-rays or radiation (that is, impenetrable to X-rays and other radiation). The radiopaque element 114 may be in the form of a radiopaque coating and/or radiopaque element 114. In some embodiments, the radiopaque coating deposits elements with a high density such as gold, tungsten, or platinum onto the nitinol material of the medical guidewire assembly 100. The coating may be applied to the guidewire assembly 100 using various techniques previously described above. In some embodiments, the radiopaque element 114 may be in the form of a radiopaque coil which may be positioned overtop of the outer diameter of the guidewire assembly 100. With a mechanical puncture device, a smooth transition may need to be created over the section of the curve with the coil to enable smooth crossing of the medical guidewire assembly 100 across the septum. This could be achieved via a PTFE liner placed over top of the coil or via a ramp of material built up in front of and behind the coil. Yet another option for the radiopaque element 114 may include creating the medical guidewire assembly 100 out of a spring-tempered stainless steel mandrel that may also enhance radiopacity and/or echogenicity of the medical guidewire assembly 100 (while not as radiopaque as tungsten, stainless steel may improve upon the visibility of nitinol).

In some embodiments, the identification device 112 may comprise an echogenic element 116 configured to be detectable by an echogenic sensor of a medical imaging system. The echogenic element 116 includes a substance having the ability to bounce an echo (such as, to return a signal in ultrasound examinations). The echogenic element 116 may be applied to the guidewire assembly 100 through various means as previously described above. The echogenic element 116 may provide (include) surface irregularities and/or the addition of elements on a portion of the medical guidewire assembly 100 of sufficiently high atomic number so as to attenuate x-rays to a greater degree than the underlying device material.

In alternative embodiments, the identification device 112 includes (for instance) a synergistic combination of: (A) a radiopaque element 114 configured to be detectable by a radiopaque sensor of a medical imaging system, and (B) an echogenic element 116 configured to be detectable by an echogenic sensor of a medical imaging system.

In some instances, having discrete groupings 124 of identification devices 112a, 112b, and 112c (as depicted in FIG. 7 and FIG. 8), may be preferred as it provides users with enhanced visibility without compromising the main body of the guidewire assembly 100. For example, when forming the echogenic element 116, surface irregularities are created through various means. In one such example, grinding of the guidewire assembly 100 may be utilized. If there is a high amount of grinding, there is a risk that the guidewire assembly 100 may get caught on the puncture hole created in the tissue or create undesired tactile feedback due to excessive grinding. Therefore, it may be more desirable to create multiple, discrete echogenic elements 116 in the configurations illustrated in FIG. 7 and/or FIG. 8, in order to balance visibility with manufacturing means. In other words, having more discrete identification devices 112a, 112b, and 112c, allows for increased visibility without compromising the guidewire assembly 100.

In accordance with the embodiment as depicted in FIG. 9, the configurations which utilize multiple identification devices 112a, 112b, and 112c (e.g., those illustrated in FIG. 7 and FIG. 8) may act as depth indicators, enabling the user to observe the positioning of the guidewire assembly 100 as it advances through the puncture 126 in the tissue.

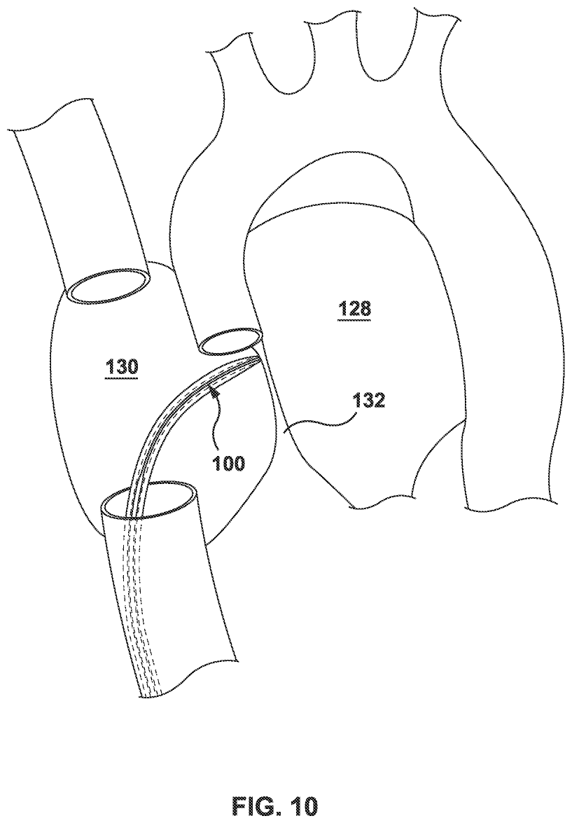

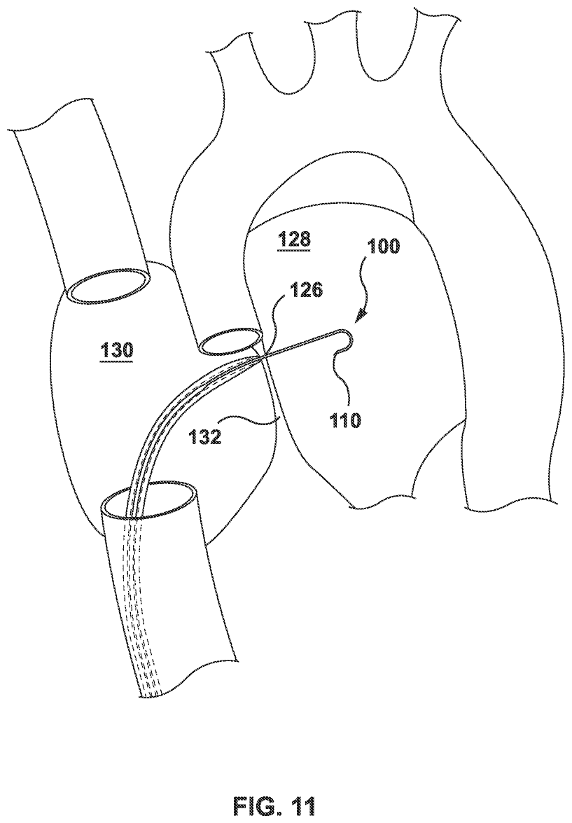

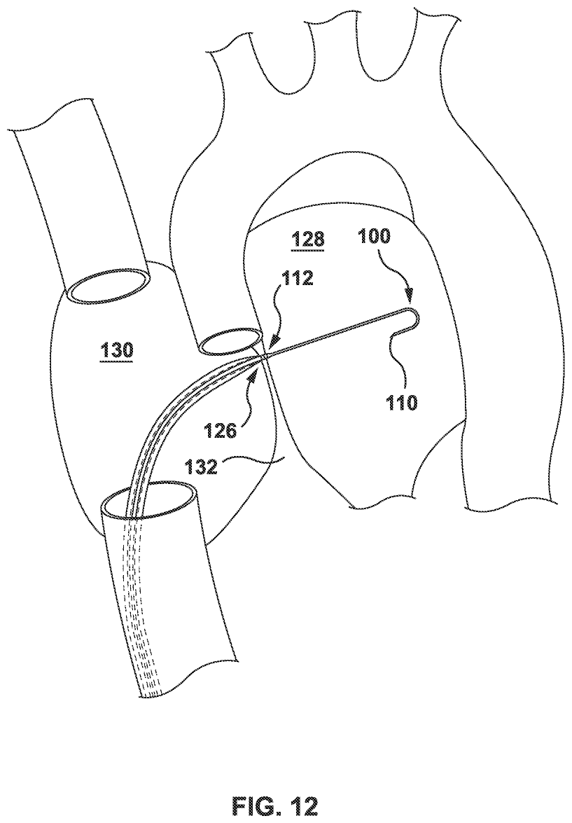

In accordance with the present invention, FIG. 10 to FIG. 12 depict a method of gaining access to the left atrium 128 of the heart, utilizing the identification devices 112 to confirm optimal positioning of the guidewire assembly 100 for facilitating the exchange of devices. First, as shown in FIG. 10, the guidewire assembly 100 is advanced into the right atrium 130 of the heart. The piercing stylet device 110 is positioned at a target location on the interatrial septum 132. With reference now to FIG. 11, the piercing stylet device 110 is advanced, forming a puncture in the interatrial septum 132. Using a medical imaging system which enables physicians to visualize the identification device 112, the guidewire assembly 100 is advanced further, through the puncture 126. The identification device 112 is positioned at a location where the outer diameter of the guidewire assembly 100 is the largest. The guidewire assembly 100 is continually advanced into the left atrium 128 until the identification device 112 is located proximate the puncture 126, as illustrated in FIG. 12. At this location, the guidewire assembly 100 is in an optimal position to provide adequate support for the exchange of ancillary devices into the left atrium 128.

The following is offered as further description of the embodiments, in which any one or more of any technical features (described in the detailed description, the summary and the claims) may be combinable with any other one or more of any technical feature (described in the detailed description, the summary and the claims). It is understood that each claim in the claims section is an open-ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the invention which does not materially modify the invention. Similarly, unless specifically made clear from its context, numerical values should be construed to include certain tolerances that the person skilled in the art would recognize as having negligible importance as they do not materially change the operability of the invention. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options would be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the invention. The foregoing has outlined the non-limiting embodiments (examples). The description is made for particular non-limiting embodiments (examples). It is understood that the non-limiting embodiments are merely illustrative as examples.

What is claimed is:

1. A medical guidewire configured for puncturing a tissue, the medical guidewire comprising:
an elongate guidewire body, wherein the elongate guidewire body comprises:
a proximal portion with a first outer diameter;
a distal portion with a second outer diameter, wherein the second outer diameter is smaller than the first outer diameter, forming a tapered section between the first outer diameter and second outer diameter;
a piercing device extending from the distal portion, forming a puncturing distal tip; and,
an identification device positioned on the elongate guidewire body, wherein the identification device is located on the first outer diameter proximal the tapered section;
whereby the identification device is configured to enhance detectable visibility of the medical guidewire.

2. The medical guidewire of claim 1, wherein the elongate guidewire body comprises a plurality of identification devices.

3. The medical guidewire of claim 2, wherein the plurality of identification devices are separated from one another at a distance between 0.1 mm and 20 mm.

4. The medical guidewire of claim 3, wherein the distance between each of the plurality of identification devices is 1.25 mm.

5. The medical guidewire of claim 2, wherein the plurality of identification devices form a discrete grouping.

6. The medical guidewire of claim 3, wherein the elongate guidewire body comprises at least two discrete groupings.

7. The medical guidewire of claim 6, wherein the plurality of identification devices are separated from one another at a distance between 0.1 mm and 20 mm and the discrete groupings are separated from one another at a distance between 1 mm and 40 mm.

8. The medical guidewire of claim 7, wherein the distance between each of the plurality of identification devices is 1.25 mm and the distance between each of the at least two discrete groupings is 10 mm.

9. The medical guidewire of claim 1, wherein the identification device has a width between 0.1 mm and 40 mm.

10. The medical guidewire of claim 9, wherein the width of the identification device is 2.5 mm.

11. The medical guidewire of claim 1, wherein the identification device is an echogenic element.

12. The medical guidewire of claim 11, wherein the echogenic element is comprised of surface irregularities on a section of the elongate guidewire body.

13. The medical guidewire of claim 12, wherein the surface irregularities are formed by laser etching, centerless grinding, or abrasive blasting, or any combination thereof.

14. The medical guidewire of claim 1, wherein the identification device is a radiopaque element.

15. The medical guidewire of claim 14, wherein the radiopaque element is a radiopaque coil.

16. The medical guidewire of claim 14, wherein the radiopaque element is formed by thermal spraying, electroplating, pad printing, or vapor deposition, or any combination thereof.

17. The medical guidewire of claim 1, wherein the identification device is a combination of an echogenic element and a radiopaque element.

18. A method of accessing a left atrium of a heart, the method comprising the steps of:
Advancing a medical guidewire into a right atrium of the heart and positioning a puncturing distal tip of the medical guidewire on a target location of a septum, the medical guidewire comprising:
an elongate guidewire body, wherein the elongate guidewire body comprises:
a proximal portion with a first outer diameter;
a distal portion with a second outer diameter, wherein the second outer diameter is smaller than the first outer diameter, forming a tapered section between the first outer diameter and second outer diameter;
a piercing device extending from the distal portion, forming the puncturing distal tip;
an identification device positioned on the elongate guidewire body, wherein the identification device is located on the first outer diameter proximal the tapered section;
Puncturing the septum and advancing the distal portion of the medical guidewire into the left atrium while visualizing the identification device on a medical imaging system; and,
Advancing the medical guidewire into the left atrium until the identification device has reached the septum, whereby the medical guidewire is in a position to facilitate and support an exchange of an ancillary device.

19. The method of claim 18, wherein the identification device is either a radiopaque element or an echogenic element or a combination of the radiopaque element and the echogenic element.

20. The method of claim 19, wherein the medical imaging system is a fluoroscopy medical-imaging system and/or an echocardiography medical-imaging system.
